(19) United States
(12) Patent Application Publication
(10) Pub. No.: US 2009/0142453 A1
(43) Pub. Date: Jun. 4, 2009

(54) Nutraceutical Coatings fo Fruits and Vegetables

(76) Inventors: George F. Lobisser, Bainbridge Island, WA (US); Carolina Andrea Torres del Campo, Yakima, WA (US); Jason F. Alexander, Yakima, WA (US)

Correspondence Address: MANN LAW GROUP, 1420 FIFTH AVENUE, SUITE 2200, SEATTLE, WA, 98101, US

(21) Appl. No.: 11/948,155

(22) Filed: Nov. 30, 2007

(51) Int. Cl. A23B 7/16; A61K 45/00

(52) U.S. Cl. 426/102; 514/789

(57) ABSTRACT

A preharvest coating for fruits, vegetables and other plants includes a primary ingredient for protecting the fruit, vegetable or plant from physical harm or damage during growing and harvesting and/or for enhancing the appearance of the fruit, vegetable or plant. In addition, the coating includes a nutraceutical component that may provide health beneficial effects to human consumers of the fruit, vegetable or plant. Such nutraceutical components can include phytonutrients such as flavonoids, stilbenes or other phytochemicals. Preferably, the nutraceutical component is included in an otherwise conventional coating and is applied by means of preharvest thermofogging of the fruit, vegetable or plant.

### FIELD OF THE INVENTION

This invention relates to the techniques of improving the nutritional and other health-related effects of fruits and vegetables. In particular, the invention contemplates improving the nutritional and other health-related effects of fruits and vegetables by applying a nutraceutical coating to the fruits and vegetables after harvest.

### BACKGROUND OF THE INVENTION

Increasingly, various foods are being recognized as having beneficial effects on human health. One particular area of interest is in phytochemicals, which, broadly, are chemical compounds derived from plants or fruits. One subset of phytochemicals are phytonutrients, which are phytochemicals or compounds that come from edible plants.

Various types of phytochemicals and phytonutrients are currently thought to have beneficial effects on human health. These include Flavonoids, Bioflavonoids, Nutraceuticals, Natural Phenolic Compounds, etc.

Flavonoids are a sub-group of phenolics that exhibit high antioxidant activity. Flavonoids are of particular interest as a nutrient or other health-beneficial agent because they have been found to reduce the risk of several ailments, including the risk of degenerative diseases, the incidence of certain cancers, the risk of cardiovascular diseases and diabetes. Flavonoids have also been shown to have other beneficial properties, including anti-inflammatory, antibacterial and anti-allergenic effects.

Heretofore, flavonoids and other phytonutrients have primarily been consumed for their beneficial effects by way of foodstuffs in which they naturally occur. To obtain the beneficial effects of flavonoids and other phytonutrients, it has been necessary to consume the foodstuffs in which they naturally occur in sufficient quantities that their beneficial effects may be realized. This requires both an adequate supply of the foodstuffs containing the naturally occurring flavonoids as well as a preference for such foodstuffs by the person seeking their beneficial effects.

Fresh fruits and vegetable are increasingly being seen as a source of beneficial nutrients. Although it has been known to apply various coatings and other agents to fruits and vegetables to assist them in growth and preserve them after harvest, no attempt has been made to incorporate flavonoids or other phytonutrients into such coatings to enhance the beneficial effects of the fruits and vegetables themselves. To the extent flavonoids or other phytonutrients have been present in such coatings, they have only been done so incidentally, and not in sufficient quantities to exhibit health-beneficial effects.

### SUMMARY OF THE INVENTION

The invention is directed to the concept of applying flavonoids or other phytonutrients to the outer surface of fruits and vegetables in concentrations sufficient to enhance promote health and possible reduce risk of disease.

Further aspects of the invention include adding flavonoids to existing types of post-harvest coatings so that fruits and vegetables treated with such coatings achieve a health-beneficial effect in addition to the other effects otherwise offered by the coatings.

Further aspects of the invention include applying a coating containing beneficial quantities of flavonoids to fruits and vegetables post harvest by means of coating, drenching, thermofogging, dipping, or spraying.

### DETAILED DESCRIPTION

As used herein, phytochemicals refers to chemical compounds derived from plants or fruits.

As used herein, phytonutrients are phytochemicals or compounds that come from edible plants.

As used herein, flavonoids comprise flavanols, flavonones, flavones, flavan-3-ols (catechins), anthocyanins and isoflavones groups.

The invention in its broader aspects includes the application of a nutraceutical coating to fruits, vegetables and other foodstuffs. Preferably, the coating includes both an “active” component in combination with an “inactive” component. For purposes of this application, the “active” component refers to a component that is included for potential health benefits to a human consumer, while the “inactive” component refers to a component that, while effective for other purposes, such as protection of the fruit from physical or environmental damage, is not intended to provide health benefits as such to the consumer.

The invention in its broader aspects includes the application of phytonutrients, such as flavonoids, to the exterior of a harvested fruit or vegetable in a quantity sufficient to have proven, likely or possible health-beneficial effects.

The invention in its broader aspects also includes the application of stilbenes to the exterior of a harvested fruit or vegetable in a quantity sufficient to have proven, likely or possible health-beneficial effects.

Preferably, the flavonoids are applied to the fruit or vegetable in the form of a coating that covers the fruit or vegetable and is consumed along with the fruit or vegetable. To this end, the coating is preferably sufficiently compatible with the texture and appearance of the fruit to be substantially unnoticeable by the consumer during the consumption.

Preferably, the flavonoids are incorporated into an existing or main coating product that will be applied to the fruits and vegetables post-harvest for other purposes, such preserving the fruit and vegetables during storage. Various such coating products are in existence and are available, for example, from Pace International, LLC of Seattle, Wash.

Generally, main coatings are applied to fruits, vegetables or other plants for purposes of providing protection (i.e., protective intent) and/or enhancing the appearance of the fruit or vegetable (i.e., cosmetic intent.) Protective intent includes protection against such things as abrasion and/or injury from processing, handling, transport; moisture loss; rapid and/or incorrect ripening; storage injury; pathogens; etc. Cosmetic intent includes such things as imparting a specific overall appearance such as high gloss, low gloss, no gloss or the appearance of no coating; enhanced color; de-emphasizing imperfections; etc.

The main coatings that are typically used for providing protection or cosmetic enhancement to fruits, vegetables or other plants include primarily naturally occurring compounds, synthetic compounds, or a combination of both. Naturally occurring compounds that are effective include waxes; oils; resins; proteins; carbohydrates (simple and complex); phospholipids; gums; and other film forming compounds of natural origin. Synthetic compounds that have been found effective include waxes; oils; proteins; carbohydrates (simple and complex); phospholipids; gums; silicones, polydimethysiloxane, and other film forming compounds of synthetic origin. Such compounds can be selected from the group including waxes, oils, resins (or combination of any film-forming compound) of natural or synthetic origin (i.e. Carnauba Wax, Beeswax, Candelilla Wax, Sunflower Oil, Olive Oil, Corn Oil, Petroleum Oil, Petrolatum, Mineral Oil, Wood Rosin, Shellac, Natural or Synthetic Resins, etc.).

In addition to the primary agents identified above, the main coatings also typically include additional agents to aid in the application of the coating and enhance its efficacy. Such additional agents include various types of surfactants, emulsifiers, wetting agents, adjuvants, carriers, suspending agents, viscosity modifiers, stabilizers and the like. Such additional agents include Alkanolamides; Alkanolamines; Alkylaryl Sulfonic Acid and Sulfonates; Alkylbenzenes; alkoxylated aryl phenols and alkylphenols; alkoxylated fatty acids, fatty esters, alcohols, and oils; Sulfonated amines and amides, Betaine Derivatives, Block Polymers, Diphenyl Sulfonate Derivatives, Alkoxylated amines and amides; Fatty esters; Fluorocarbon-based surfactants; Imidazoline Derivatives; Lanolin-based Derivatives; Lecithin and Lecithin Derivatives; Lignin and Lignin Derivatives; Succinic Anhydrides; Sulfosuccinates and Derivatives; Olefin Sulfonates; Phosphate Esters; Phosphorous Organic Derivatives; Polymeric (Polysaccharides, Acrylic Acid, Acrylamide); Protein-based Surfactants; Silicone-based Surfactants; Sorbitan Derivatives; Sucrose and Glucose esters and derivatives; Sulfates and Sulfonates of alkoxylated alkylphenols, Oils, fatty acids, alcohols, alkoxylated alcohols, dodecyl and tridecylbenzenes, Naphthalene and alkylnapthalene, petroleum; dodecyl and tridecylsulfonic acids.

To provide the nutraceutical coating, one or more active compounds are added to the main protective and/or cosmetic coating. Such active compounds are preferably of natural plant origin and are selected from phytochemicals believed to have beneficial effects on human health when consumed. Such active compounds include Phytochemicals, Bioflavanoids, Nutraceuticals, Natural Phenolic Compounds, etc. These are non-nutritive chemical compounds of natural plant origin showing numerous protective and disease preventative properties in the human body. These chemicals are produced by plants for responsive protection, and appear to have numerous benefits to health and the human body.

In practice, each of the three components described above can be combined in various proportions and mixed with a suitable liquid carrier, such as water. The proportions themselves are not critical, and each of the ingredients can comprise anywhere from 0.05 to 99.5 percent of the final mixture.

Preferably, the coating containing the flavonoids is applied to the exterior of the fruit or vegetable by any of several known means of applying such coatings. Such means include thermofogging, dipping, drenching or spraying. Alternatively, the flavonoids can be directly incorporated into the food as an alternate embodiment.

As used herein, thermofogging refers to a technique by which coatings or other agents may be applied to fruit by atomizing the agent and entraining it in a flow of heated air at controlled temperatures, concentrations and velocities. Various forms of thermofogging techniques are described, for example, in U.S. Pat. No. 6,723,364, the specification of which is incorporated by reference herein.

Thermofogging the flavonoid containing coating has the additional advantage that repeated applications, particularly while the fruit or vegetable remains in storage, are possible. This has the effect of increasing the total flavonoid content of the fruit when it ultimately reaches and is consumed by the consumer.

In another aspect, another group of compounds known as stilbenes can also be applied to the fruit, both with or without the co-presence of flavonoids. The stilbenes can similarly be incorporated into an existing coating product and can similarly be applied to the fruit through such means as thermofogging, dipping, drenching or spraying. Alternatively, the stilbenes can be directly incorporated into the food as an alternate embodiment.

Although the concept herein described has been considered primarily in the context of post-harvest applications, it will be appreciated that flavonoid or stilbenes containing coatings or preparations can also be applied to fruits and vegetables pre-harvest as well. and that in its broader aspects, the concept is not limited solely to post-harvest applications.

It will be appreciated that, while particular concentrations, temperatures, velocities, times, etc. have been described, other parameters can be advantageously selected without deviating from the invention in its broader aspects.

What is claimed is:

1. A coating for preharvest application to an edible fruit, plant or vegetable, said coating comprising a first agent for providing protective and/or cosmetic enhancement to the fruit, plant or vegetable and a second agent comprising a phytochemical for providing health beneficial effects.

2. A coating as defined in claim 1 wherein said second agent is a phytonutrient.

3. A coating as defined in claim 2 wherein said phytonutrient is selected from the group consisting of Flavonoids, Bioflavanoids, Nutraceuticals, Natural Phenolic Compounds, and Stilbenes.

4. A coating as defined in claim 3 wherein coating is applied to the fruit, plant or vegetable by means of thermofogging.

5. A coating as defined in claim 3 wherein coating is applied to the fruit, plant or vegetable by means of drenching.

6. A coating as defined in claim 3 wherein coating is applied to the fruit, plant or vegetable by means of spraying.)

7. A coating as defined in claim 1 wherein said coating further includes one or more additional agents to enhance the efficacy of the coating.

8. A coating as defined in claim 7 wherein the additional agent is selected from the group consisting of surfactants, emulsifiers, wetting agents, adjuvants, carriers, suspending agents, viscosity modifiers and stabilizers.

9. A coating as defined in claim 7 wherein the additional agent is selected from the group consisting of Alkanolamides; Alkanolamines; Alkylaryl Sulfonic Acid and Sulfonates; Alkylbenzenes; alkoxylated aryl phenols and alkylphenols; alkoxylated fatty acids, fatty esters, alcohols, and oils; Sulfonated amines and amides, Betaine Derivatives, Block Polymers, Diphenyl Sulfonate Derivatives, Alkoxylated amines and amides; Fatty esters; Fluorocarbon-based surfactants; Imidazoline Derivatives; Lanolin-based Derivatives; Lecithin and Lecithin Derivatives; Lignin and Lignin Derivatives; Succinic Anhydrides; Sulfosuccinates and Derivatives; Olefin Sulfonates; Phosphate Esters; Phosphorous Organic Derivatives; Polymeric (Polysaccharides, Acrylic Acid, Acrylamide); Protein-based Surfactants; Silicone-based Surfactants; Sorbitan Derivatives; Sucrose and Glucose esters and derivatives; Sulfates and Sulfonates of alkoxylated alkylphenols, Oils, fatty acids, alcohols, alkoxylated alcohols, dodecyl and tridecylbenzenes, Naphthalene and alkylnapthalene, petroleum; dodecyl and tridecylsulfonic acids.

10. A coating as defined in claim 1 wherein said first agent is a naturally occurring compound selected from the group consisting of waxes; oils; resins; proteins; carbohydrates (simple and complex); phospholipids and gums.

11. A coating as defined in claim 1 wherein said first agent is a synthetic compound selected from the group consisting of waxes; oils; proteins; carbohydrates (simple and complex); phospholipids; gums; silicones, and polydimethysiloxane.

12. A coating as defined in claim 1 wherein said first agent is selected from the group consisting of Carnauba Wax, Beeswax, Candelilla Wax, Sunflower Oil, Olive Oil, Corn Oil, Petroleum Oil, Petrolatum, Mineral Oil, Wood Rosin, Shellac, Natural and Synthetic Resins.

13. A food product comprising a fruit, plant or vegetable having a coating applied to the exterior thereof, said coating including a first agent for providing protective and/or cosmetic enhancement to the fruit, plant or vegetable and a second agent comprising a phytochemical for providing health beneficial effects.

14. A food product as described in claim 13 wherein said coating is applied post harvest.

15. A food product as described in claim 13 wherein said second agent is a phytonutrient selected from the group consisting of Flavonoids, Bioflavanoids, Nutraceuticals, Natural Phenolic Compounds, and Stilbenes.

16. A method of treating a fruit, plant or vegetable comprising the steps of forming a coating material having a first a first agent for providing protective and/or cosmetic enhancement to the fruit, plant or vegetable and a second agent comprising a phytochemical for providing health beneficial effects, and applying said coating material to the fruit, plant or vegetable to form a protective and nutritional coating on the fruit, plant or vegetable.

17. A method as described in claim 16 wherein the coating material is applied to the fruit, plant or vegetable post harvest.

18. A method as described in claim 17 wherein the coating is applied by means of thermofogging.

19. A method as described in claim 17 wherein the coating is applied by means of drenching.

20. A method as described in claim 17 wherein the coating is applied by means of spraying.
